(19) United States
(12) Patent Application Publication
(10) Pub. No.: US 2015/0196457 A1
(43) Pub. Date: Jul. 16, 2015

(54) AMPOULE SYSTEM WITH MEDICAL LIQUID AND CAP WITH FILTER FACILITY

(71) Applicant: HERAEUS MEDICAL GMBH, Wehrheim (DE)

(72) Inventor: EDGAR WÜST, GROSS -UMSTADT (DE)

(73) Assignee: HERAEUS MEDICAL GMBH, WEHRHEIM (DE)

(21) Appl. No.: 14/585,725

(22) Filed: Dec. 30, 2014

(30) Foreign Application Priority Data
Jan. 10, 2014 (DE) 10 2014 200 286.9

(51) Int. Cl. A61K 6/08; A61J 1/14; A61L 24/06; A61J 1/06; A61B 17/88

(52) U.S. Cl. CPC A61J 1/1456; A61J 1/065; A61J 1/1412; A61B 17/8833; A61L 24/06; A61B 2017/8838

(57) ABSTRACT

An ampoule system comprising an ampoule (1) made of glass and enclosing an internal space, the ampoule having an ampoule body (4) and an ampoule head (5) that can be broken off from the ampoule body (4) to open up the internal space, the internal space of the ampoule (1) containing a liquid (7) for a medical application or for producing a mixture for a medical application, wherein the ampoule system comprises a cap (2) that can be plugged onto the ampoule body (4) such that the opening of the internal space from the ampoule head (5) being broken off is covered by the cap (2), and the cap (2) comprises a filter facility (3), whereby the filter facility (3) is at least one sieve (3) and/or at least one filter and the liquid (7) can be poured through the filter facility (3) from the internal space from the ampoule (1).

The invention relates to an ampoule system comprising an ampoule made of glass, whereby the ampoule encloses an internal space and comprises an ampoule body and an ampoule head that can be broken off, whereby the ampoule head can be broken off from the ampoule body in appropriate manner such that the internal space opens up by the ampoule head being broken off, and whereby the internal space of the ampoule contains a liquid for a medical application or for producing a mixture for a medical application, as well as a method for use of said ampoule.

### BACKGROUND OF THE INVENTION

Ampoule systems are used in medicine for sterile and safe storage of liquids for application in medicine. Ampoules of this type are used in medicine, inter alia, for storage of a starting component of a bone cement, in particular in the application of storing a liquid methylmethacrylate as monomer for forming a bone cement made of polymethylmethacrylate (PMMA). Said PMMA bone cements are used in medicine for permanent mechanical fixation of total joint endoprostheses. The redox initiator systems used for this purpose usually consist of peroxides, accelerators and, if applicable, suitable reducing agents. Radicals are formed only if all components of the redox initiator systems act in concert. For this reason, the components of the redox initiator system are arranged appropriately in the separate containers such that these cannot trigger a radical polymerisation. Only when the two components are mixed to produce a cement dough, the components of the previously separated redox initiator system react with each other forming radicals which trigger the radical polymerisation of the at least one monomer. The radical polymerisation then leads to the formation of polymers while consuming the monomer, whereby the cement dough is cured. During many surgeries, the components are mixed in a bowl, in which both components are placed and then mixed manually. In this context, a monomer, in liquid form, is taken from an ampoule made of glass after breaking the ampoule.

Vacuum cementing systems, in which said ampoules are used, are known from the prior art. For example, DE 10 2010 026 497 A1, being generic, discloses a vacuum cementing system, in which an ampoule head is being broken off and an ampoule can be opened by this means. A negative pressure is used to draw the liquid from the ampoule and/or a storage container for the ampoule through a tubing into a cement powder or a cement paste. A sieve can be arranged in said tubing to retain any splinters from the ampoule.

EP 0 926 500 A2 discloses a device for removal of a liquid from a glass ampoule that is well-suited for shearing off the ampoule head and removing the liquid from the opened ampoule through a cannula, in which a filter can be arranged. Another complex structure of this type for opening and emptying an ampoule is known from WO 99/37256 A1.

Structures of this type are too complex and involved for many applications. Often the starting components of bone cements are simply mixed in a bowl by the user at the quantity desired by the user and applied with a spatula during the surgery. However, it shall still be possible to remove the monomer from glass ampoules for this purpose. It can be advantageous in this context that the user does not use up the entire content of an ampoule, but rather can add a desired quantity of the monomer for mixing a user—provided quantity of the cement powder or cement paste with the monomer. This application also involves the use of said glass ampoules, whereby there is an inherent risk during the production of the bone cement, i.e. glass splinters generated while the ampoule is being opened might enter into the bone cement mixture along with the liquid. Said glass splinters can weaken the bone cement. They can also have a disadvantageous effect on the healing process of the patient.

Accordingly, it is the object of the invention to overcome the disadvantages of the prior art. Specifically, an ampoule system and a method for application thereof is to be developed, in which glass ampoules of simple structure can be used without glass splinters impairing the medical liquid contained in the glass ampoules. Moreover, it shall be possible to use any amount of liquid for the medical use without glass splinters contaminating the liquid and without the structure becoming too involved. The structure shall also be as easy as possible to operate. It needs to be noted in this context that the use of medical liquids in the field of surgery often requires safe and reliable sterilisation of all parts contacting the patient or the treatment means. The application of the method according to the invention and the ampoule system shall minimise the possibilities of misuse. Moreover, the ampoule system shall be as inexpensive to manufacture as possible since the ampoule systems are usually disposed of after single use for reasons of hygiene.

### SUMMARY OF THE INVENTION

The objects of the invention are met by an ampoule system comprising an ampoule made of glass, whereby the ampoule encloses an internal space and comprises an ampoule body and an ampoule head that can be broken off, whereby the ampoule head can be broken off from the ampoule body in appropriate manner such that the internal space opens up by the ampoule head being broken off, and whereby the internal space of the ampoule contains a liquid for a medical application or for producing a mixture for a medical application, whereby the ampoule system comprises a cap that can be plugged onto the ampoule body in appropriate manner such that the opening of the internal space from the ampoule head being broken off is covered by the cap, and the cap comprises a filter facility, whereby the filter facility is at least one sieve and/or at least one filter and the liquid can be poured through the filter facility from the internal space from the ampoule.

Preferably, the liquid is a methylmethacrylate. It is particularly preferred to use the methylmethacrylate for producing a polymethylmethacrylate bone cement (PMMA bone cement).

### DETAILED DESCRIPTION

Common silicon dioxide-based glasses, as well as other materials, are conceivable as glass for the ampoule. Basically, all glasses that do not undergo a chemical reaction with the content and can be broken open without major difficulties are well-suited for use for the glass ampoules.

It can be particularly preferred, according to the invention, that the liquid can be poured out from the internal space from the ampoule through the filter facility exclusively.

The use of multiple sieves and/or filters arranged downstream with regard to the flow direction of the liquid is advantageous in that glass particles of different sizes are trapped in different sieves and/or filters and the free cross-section for the liquid to flow through stays larger as compared to the use of only one filter or sieve.

Regardless, the use of just one sieve is preferred according to the invention, since this simplifies the structure and breaking the ampoules open usually generates only few glass splinters that need to be retained.

Ampoule systems according to the invention can be provided appropriately such that the cap touches against the ampoule body by its entire circumference when the cap is plugged onto the ampoule and the ampoule head is broken off.

Preferably, the invention can just as well provide the cap to touch against the ampoule body in a press-fit and/or in fluid-tight manner when the cap is plugged onto the ampoule and the ampoule head is broken off.

This can ensure that the liquid can be poured from the internal space of the ampoule through the filter facility exclusively, and not past the cap.

A particularly preferred embodiment of the invention proposes the ampoule system to consist of the cap and the ampoule containing the liquid.

Said ampoule system then has no further parts other than the ampoule containing the liquid and the cap. The ampoule system comprising said small number of parts is sufficient for manual mixing of a bone cement or for individual use of the liquid, and therefore has a simple and inexpensive structure.

The invention can provide the cap to be attached or secured in detachable manner to the end of the ampoule body opposite from the ampoule head.

This allows the ampoule system to be sterilised as an entity without the ampoule or the cap having to be treated individually as individual parts or to be individually placed in the device for sterilisation of the ampoule system. Accordingly, the cap assumes said position before the ampoule head is or is being broken off the ampoule body.

An embodiment, in which possible glass splinters are retained particularly well, results if the invention provides the side of the cap, which faces the opening of the internal space when the ampoule head is broken off and when the cap is plugged on, to be connected to the other side of the cap, which faces the surroundings of the ampoule system, only by means of the filter facility and these to be separate otherwise.

This ensures and/or makes clear that no further openings and/or bushings are to be present in the cap through which the liquid plus glass splinters that might be present can flow through the cap. This, in turn, ensures that the glass splinters present in the liquid or at least those from a certain size are fully removed from the liquid by means of the sieve and/or the filter.

An embodiment of the invention that can be implemented particularly simply and inexpensively can be attained in that the cap comprising the filter facility consists of a plastic material, preferably of a plastic material that is not reactive with methylmethacrylate, particularly preferably of polypropylene.

This renders the manufacture of the cap easy and inexpensive to implement. For example, and preferred according to the invention, the cap can be manufactured by means of an injection moulding process. Alternatively, the invention can just as well provide the sieve or sieves and/or the filter or filters to consist of a different material (for example a metal or a textile material) and the rest of the cap to consist of plastic material. In this case, the filter facility/facilities of said caps are inserted in or attached to the rest of the cap.

The invention can provide the sieve to have a mesh width between 0.1 mm and 0.5 mm or the sieves to have a mesh width between 0.1 mm and 0.5 mm. Preferably, the invention can provide the sieve to have a mesh width between 0.2 mm and 0.3 mm.

Said mesh widths allow glass splinters that are usually generated while breaking off the ampoule head, to be filtered out and/or removed, while still providing for a high or still sufficient flow rate of the liquid through the sieve or sieves. The specified mesh widths can be the average size of the mesh widths.

The objects of the present invention are also met by a method for use of an ampoule system as specified above, in which the ampoule head is broken off from the ampoule body while opening the internal space of the ampoule, followed by plugging the cap onto the ampoule over the opening of the opened ampoule, and subsequently pouring the liquid through the filter facility from the internal space of the ampoule.

Preferably, the invention can provide the cap to be plugged over the opening of the opened ampoule onto the ampoule body of the ampoule.

The invention can just as well provide the cap, prior to breaking off the ampoule head, to be plugged onto the ampoule and both to be sterilised together, and the cap to be pulled off the side of the ampoule bearing the ampoule head prior to breaking off the ampoule head or the cap to be pulled off the side of the ampoule opposite from the ampoule head after breaking off the ampoule head.

Said methods ensure that the cap is always used together with the ampoule and cannot get lost or need to be searched for unless it is used incorrectly. Moreover, this can ensure that both the cap and the ampoule are getting sterilised and neither one of the two is inadvertently not subjected to sterilisation.

Alternatively, the invention could just as well provide the cap to be connected to the ampoule body by means of a connection, for example by means of a fin. For this purpose, a loop around the ampoule body could secure the cap to the ampoule body by means of the fin. The cap is then plugged on by bending the fin over the opening on the ampoule body.

A particularly preferred embodiment of the method can provide that the liquid, after pouring it out, is mixed with a powder or a paste for producing a cement, preferably for producing a bone cement, particularly preferably that the liquid is a methylmethacrylate and is mixed with a powder or a paste for producing a PMMA bone cement.

The invention is based on the surprising finding that it is feasible, through the use of a cap comprising at least one sieve or at least one filter, to retain glass splinters, which can be generated upon the glass ampoule being opened, in inexpensive and simple manner. Since the cap comprising the filter facility can also be plugged onto the opposite end of the ampoule, both can be sterilised together without either one of the parts (probably the cap in the majority of cases) being forgotten and not being subjected to sterilisation. During the use of an ampoule system according to the invention, the ampoule can be opened and then the cap comprising the filter facility can simply be plugged over the opening thus generated. The opened ampoule is then ready for use.

### BRIEF DESCRIPTION OF THE DRAWINGS

Further exemplary embodiments of the invention shall be illustrated in the following on the basis of five schematic figures, though without limiting the scope of the invention. In the figures:

FIG. 1: shows a schematic perspective view of an ampoule system according to the invention;

FIG. 2: shows a schematic cross-sectional view of a further ampoule system according to the invention in its starting state;

FIG. 3: shows a schematic cross-sectional view of the ampoule system according to FIG. 2 with the ampoule opened;

FIG. 4: shows a schematic cross-sectional view of the ampoule system according to FIG. 2 with the ampoule opened and the cap plugged over the opening; and

FIG. 5: shows a schematic cross-sectional view of the ampoule system according to FIG. 4, with the liquid being poured from the internal space from the ampoule.

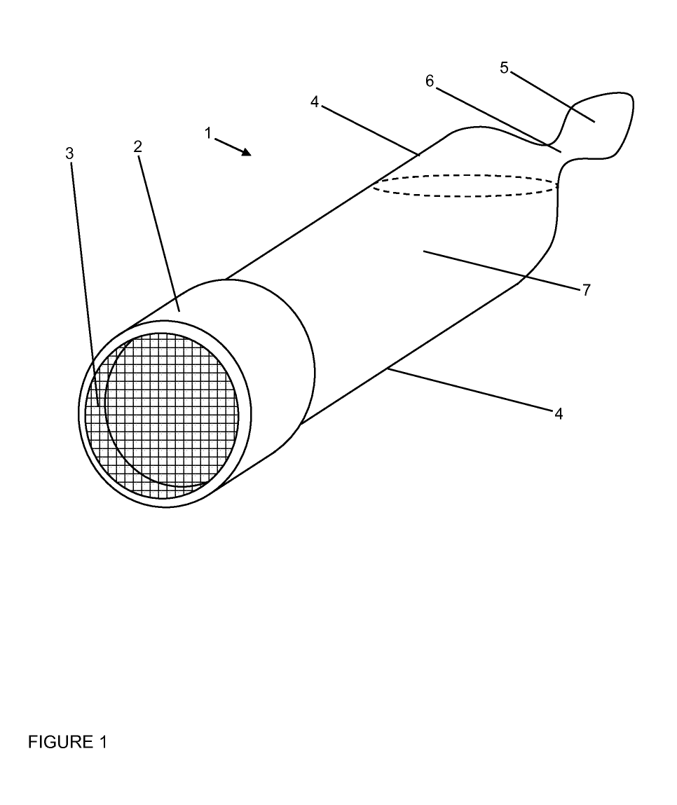

FIG. 1 shows a schematic perspective view of an ampoule system according to the invention comprising an ampoulel made of glass and a cap 2 made of plastic material. Cap 2 is a plastic tube that is closed on one side by a sieve 3. The sieve 3, which can also consist of plastic material, has an average mesh width of 0.25 mm at a standard deviation of +/−0.05 mm.

The ampoule 1 consists of an ampoule body 4 and an ampoule head 5 connected to each other by means of an ampoule neck 6. The ampoule 1 can be opened by breaking off the ampoule head 5 from the ampoule body 4. The ampoule 1 breaks at the pre-determined breakage site on the ampoule neck 6 in this context. A liquid 7 for use in a medical application is situated on the inside of the ampoule 1. The liquid 7 does not fill the ampoule 1 completely. There also is a supernatant gas (on the top in FIG. 1) present in the ampoule 1. The boundary between the liquid 7 and the supernatant gas is shown as a dashed line in FIG. 1. The ampoule 1 contains, for example, vaccines, calcium solutions or a liquid starting component for a bone cement as liquid 7.

The cap 2 is plugged into the side of the ampoule 1 opposite from the ampoule head 5. The base of the ampoule 1 can be seen through the mesh of the sieve 3 in FIG. 1. The internal diameter of the cap 2 is selected appropriately such that it can be plugged onto the ampoule 1 from both sides and the cap sits in this position in a press-fit. Any remaining gaps are preferably smaller than or at most equal in size to the mesh size of the sieve 3. The ampoule body 4 is cylindrical in shape and has a decreasing diameter on the ampoule neck 6 and a rounded closed end on the ampoule base. The thickness of the wall of the ampoule 1 is homogeneous (within common production tolerances) and can be thinner at the ampoule neck 6 in order to favour the ampoule 1 breaking at the ampoule neck 6.

For a use of the ampoule system for producing a PMMA bone cement, the ampoule system, the way it is shown in FIG. 1, is placed in a sterilisation facility or sterilisation chamber for sterilisation. A sterilising gas is used to sterilise both the ampoule 1 and the cap 2 comprising the sieve 3. It is self-evident that a sterilisation can take place in any other application as well.

The ampoule system is used after sterilisation or without sterilisation. The ampoule head 5 is broken off manually. Then the cap 2 is pulled off the ampoule base and plugged onto the ampoule body 4 on the other side, over the opening in the ampoule neck 6. The liquid content 7 of the ampoule 1 is emptied from the ampoule through the sieve 3. All glass splinters, which were generated while breaking off the ampoule head 5 and entered into the liquid 7, are retained by the sieve 3 while the liquid is poured out.

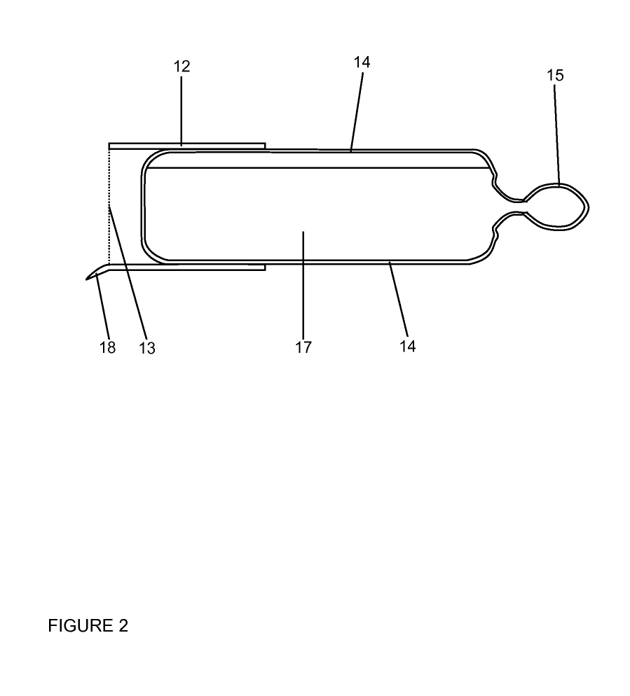

FIGS. 2 to 5 show schematic cross-sectional views of ampoule systems according to the invention that show the work flow of a method according to the invention and the function of the ampoule system according to the invention. FIG. 2 shows a schematic cross-sectional view of an ampoule system according to the invention in its starting state. A cap 12 comprising a grid-shaped sieve 13 is plugged onto the ampoule base of the ampoule. The ampoule itself is subdivided into an ampoule body 14 and an ampoule head 15 that are connected to each other by being a single part and form the border of a joint internal space that contains a liquid methylmethacrylate 17 as liquid 17 for medical purposes, namely for producing a PMMA bone cement. The surface of the liquid 17 is indicated as a horizontal line in FIG. 2. A spout 18 is provided on the cap 12 to make the liquid 17 easier to pour out. The spout 18 need not be provided, but is advantageous for the use of the ampoule system in that the liquid 17 can be poured out at high precision without dripping or without residual liquid 17 flowing down the outside of the cap 12, provided the shape of the spout 18 is adequate.

The ampoule system in the position shown in FIG. 2 is initially sterilised with a sterilising gas. Both the ampoule and the cap 12 are sterilised in this process. Subsequently, the ampoule system is ready for use during a surgery.

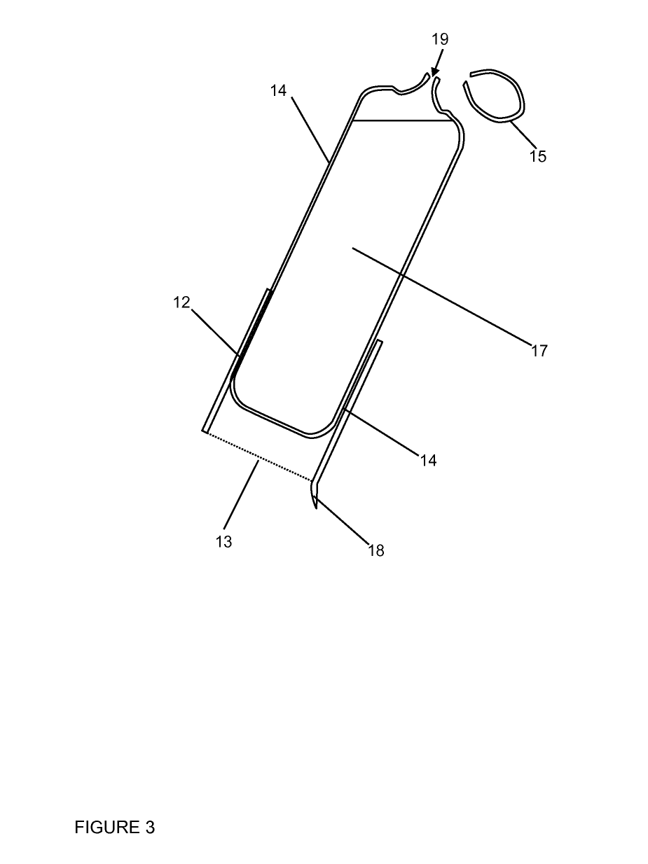

To open the ampoule, the ampoule head 15 is broken off manually from the ampoule body 14. This state is shown in a schematic cross-sectional view in FIG. 3. Obviously, the ampoule is held appropriately in the process such that the liquid 17 does not leak from the ampoule. The ampoule then has an opening 19 on top through which the liquid 17 can be poured from the ampoule. Breaking off the ampoule, small glass splinters (not shown) may detach from the site of breakage and drop into the liquid 17.

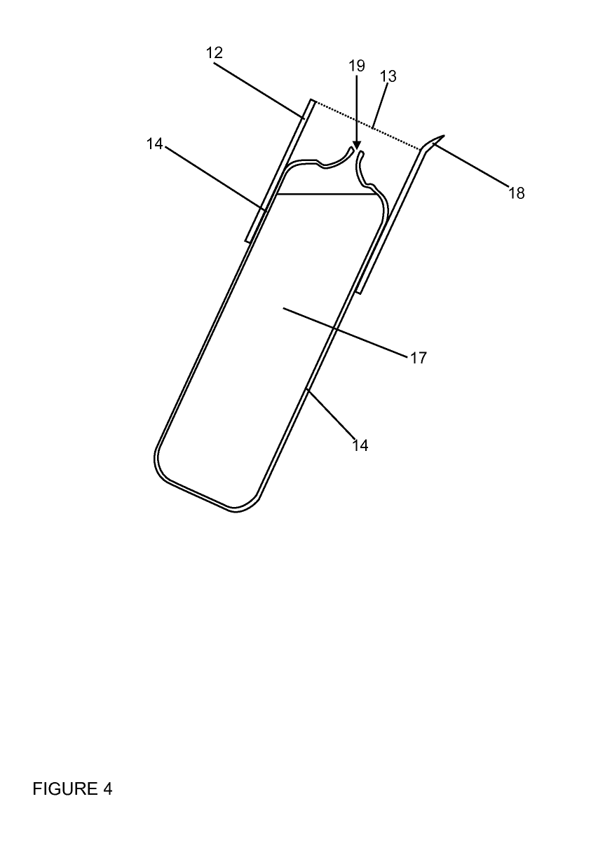

To prevent these glass splinters from being processed along with the liquid, the cap 12 is then plugged onto the other side of the ampoule, namely over the opening 19. This situation is shown in FIG. 4. The internal walls of the cap 12 are shaped appropriately such that they close off, in circumferential and form-fitting manner, the outer circumference of the ampoule body 14, which is shaped cylindrical in some region(s). The material from which the cap 12 is made (preferably an injection moulded part made of plastic material) possesses the requisite elasticity and deformability such that the cap 12 can be plugged easily onto the ampoule body 14 and is held in place on the ampoule body 14 by means of the press-fit of the cap 12. The single passage of the cap 12, and therefore of the ampoule system, is covered by the sieve 13 and/or the grid 13. The liquid 17 can be poured from the ampoule only through the grid 13 and/or the sieve 13.

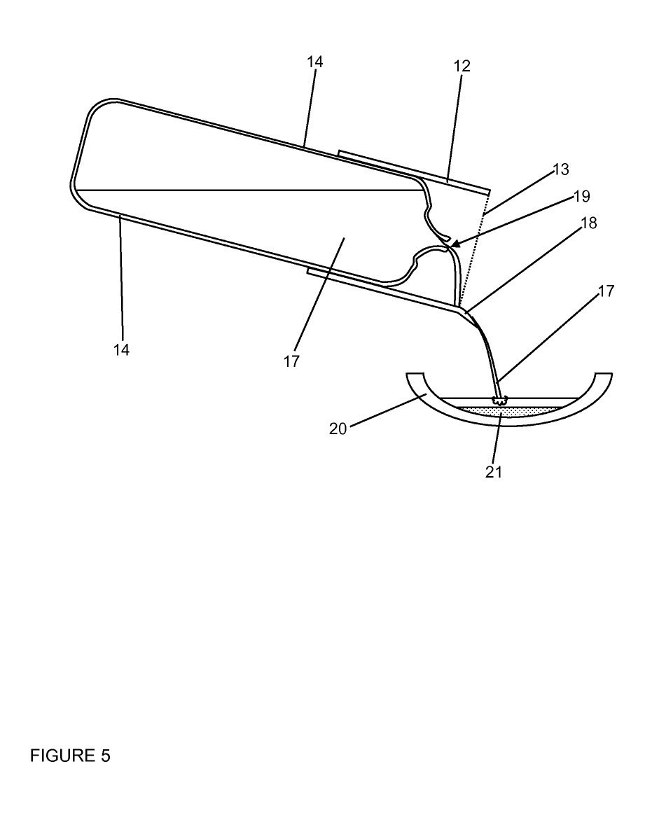

The situation during the pouring out is shown in a schematic depiction as a cross-section in FIG. 5. The liquid 17 is poured from the ampoule through the sieve 13 and/or the grid 13 via the spout 18 into a bowl 20, in which a powder 21 is situated as the second component of the PMMA bone cement. The liquid 17 and the powder 21 can be mixed in the bowl 20 by means of a spatula and the ready-made stirred bone cement is then available for use. The grid 13 and/or the sieve 13 have a mesh width between 0.2 mm and 0.3 mm and therefore retain glass splinters that exceed this size.

This prevents these glass splinters from inadvertently entering into the bone cement and impairing the bone cement or its usability.

The features of the invention disclosed in the preceding description and in the claims, figures, and exemplary embodiments, can be essential for the implementation of the various embodiments of the invention both alone and in any combination.

### LIST OF REFERENCE NUMBERS

1 Ampoule
2, 12 Cap
3, 13 Sieve
4, 14 Ampoule body
5, 15 Ampoule head
6 Ampoule neck
7, 17 Liquid
18 Spout
19 Opening
20 Bowl
21 Powder

1. Ampoule system comprising an ampoule (1) made of glass, the ampoule (1) enclosing an internal space and having an ampoule body (4, 14) and an ampoule head (5, 15) that can be broken off from the ampoule body (4, 14) to open the internal space, the internal space of the ampoule (1) containing a liquid (7, 17) for a medical application or for producing a mixture for a medical application, wherein
the ampoule system comprises a cap (2, 12) that can be plugged onto the ampoule body (4, 14) in such manner such that the opening (19) of the internal space formed by the ampoule head (5, 15) being broken off is covered by the cap (2, 12), and the cap (2, 12) comprises a filter facility (3, 13), whereby the filter facility (3, 13) is at least one sieve (3, 13) and/or at least one filter and the liquid (7, 17) can be poured through the filter facility (3, 13) from the internal space of the ampoule (1).

2. Ampoule system according to claim 1, wherein
the cap (2, 12) touches against the ampoule body (4, 14) by its entire circumference when the cap is plugged onto the ampoule (1) and the ampoule head (5, 15) is broken off.

3. Ampoule system according to claim 1, wherein the ampoule system is comprised of the cap (2, 12) and the ampoule (1) containing the liquid (7, 17).

4. Ampoule system according to claim 1, wherein the cap (2, 12) is attached or secured in detachable manner to the end of the ampoule body (4, 14) opposite from the ampoule head (5, 15).

5. Ampoule system according to claim 1, wherein the side of the cap (2, 12), which faces the opening (19) of the internal space when the ampoule head (5, 15) is broken off and when the cap (2, 12) is plugged on, is connected to the side of the cap (2, 12) which faces the surroundings of the ampoule system, only by means of the filter facility (3, 13) and these are separate otherwise.

6. Ampoule system according to claim 1, wherein the cap (2, 12) comprising the filter facility (3, 13) is comprised of a plastic material.

7. Ampoule system according to claim 1, wherein the sieve (3, 13) has a mesh width between 0.1 mm and 0.5 mm or the sieves (3, 13) have a mesh width between 0.1 mm and 0.5 mm.

8. Method for removing the liquid contained by the ampoule system of claim 1, wherein the ampoule head (5, 15) is broken off from the ampoule body (4, 14) while opening the internal space of the ampoule (1), followed by plugging the cap (2, 12) onto the ampoule (1) over the opening (19) of the opened ampoule (1), and subsequently pouring the liquid (7, 17) through the filter facility (3, 13) from the internal space of the ampoule (1).

9. Method according to claim 8, wherein the cap (2, 12), prior to breaking off the ampoule head (5, 15), is plugged onto the ampoule (1) and both are sterilized together, and the cap (2, 12) is pulled off the side of the ampoule (1) bearing the ampoule head (5, 15) prior to breaking off the ampoule head (5, 15) or the cap (2, 12) is pulled off the side of the ampoule (1) opposite from the ampoule head (5, 15) after breaking off the ampoule head (5, 15).

10. Method according to claim 9, wherein the liquid (7, 17), after being poured out of the ampoule, is mixed with a powder (21) or a paste for producing a cement.
